(19) United States
(12) Patent Application Publication
(10) Pub. No.: US 2014/0276389 A1
(43) Pub. Date: Sep. 18, 2014

(54) SELECTIVE GRIP DEVICE FOR DRIVE MECHANISM

(71) Applicant: Sean Walker, Mountain View, CA (US)

(72) Inventor: Sean Walker, Mountain View, CA (US)

(21) Appl. No.: 13/801,957

(22) Filed: Mar. 13, 2013

(51) Int. Cl. A61M 25/01

(52) U.S. Cl. CPC A61M 25/0105
USPC 604/95.01

(57) ABSTRACT

A gripping device includes a housing having an outer periphery and a lumen wall arranged within the housing. The lumen wall may define a passage and the passage may be configured to receive an instrument. The lumen wall may have a flexible inwardly facing surface configured to grip the instrument in response to receiving a grip signal and release the instrument in response to receiving a release signal.

### BACKGROUND

Robotic surgical systems and devices are well suited for use in performing minimally invasive medical procedures as opposed to conventional techniques that require large incisions to permit the surgeon's hands access into the patient's body cavity. Advances in technology have led to significant changes in the field of medical surgery such that minimally invasive surgeries (MIS) have become increasingly popular.

As opposed to traditional open surgery, MIS may typically be performed by entering the body through the skin, blood vessels, gastrointestinal tract, or an anatomical opening utilizing small incisions made on the patient's body. However, such procedures (e.g., endovascular, laparoscopic, arthroscopic, coronary, etc.) require manipulation and control over a variety of devices, ranging from guidewires and microcatheters to balloons and stents.

In order to manipulate the medical instruments (e.g., a guidewire), medical professionals have traditionally used devices which allow the professional to apply a torque to a guidewire while inside the patient's body. Torqueing the guidewire allows the medical professional to change the spatial orientation of the tip of the guidewire while maneuvering inside the patient's anatomy, e.g., to insert or retract the guidewire, or to rotate the guidewire. As the guidewire advances into the patient's body, the length of the guidewire outside the patient decreases and control of the guidewire becomes increasingly difficult due to the shortened length of guidewire available for manipulating the guidewire.

Many of the commercially available torque devices require the medical professional to pause the procedure, loosen the torque device, reposition the device proximally along the guidewire to provide additional length between patient's body and torque device, and then tighten the device to secure its position. This process of loosening and repositioning may occur multiple times during a medical procedure. Due to the complexities of MIS procedures, for example robotically controlled endovascular procedures, manipulation and control is required over a variety of medical devices (e.g., guidewires, stents, etc.). As a result, it is often challenging to advance or retract a full variety of medical instruments required by robotic surgical systems during medical procedures.

As such, there is a need for a torque device and system that may be easily interchanged with the robotic system yet allow for the practitioner to customize the torque device's grip to a wide variety of medical devices, while also providing continuous insertion and rotation of the medical device into a patient.

### SUMMARY

An exemplary gripping device may include a housing having an outer periphery and a lumen wall arranged within the housing. The lumen wall may define a passage configured to receive an instrument. The lumen wall may have a flexible inwardly facing surface configured to grip the instrument in response to receiving a grip signal and release the instrument in response to receiving a release signal.

In another exemplary illustration, an elongate device drive mechanism includes a first gripping device having a first housing and a first lumen arranged within the first housing. The first lumen may be configured to receive a first portion of an instrument. The drive mechanism may include a second gripping device having a second housing and a second lumen arranged within the second housing. The second lumen may be configured to receive a second portion of the instrument. The second gripping device may be spaced and moveable along an axis with respect to the first gripping device. Each of the first lumen and the second lumen may include a lumen wall with a flexible inwardly facing surface configured to selectively grip the respective portion of the instrument.

### BRIEF DESCRIPTION

FIG. 1 is a perspective view of an exemplary robotic catheter assembly;

FIG. 2 is a side view of an exemplary torque system for the robotic catheter assembly;

FIG. 3 is an exemplary torque device of the torque system;

FIG. 4 is another exemplary torque device of the torque system;

FIG. 5A is a cross-sectional view of the torque device having a released actuator;

FIG. 5B is a cross-sectional view of the torque device having an actuator gripping an exemplary instrument;

FIG. 6A is a side view of another exemplary torque device having two connected housings;

FIG. 6B is a cross-sectional view of the exemplary torque device having two connected housings;

FIG. 7 is a perspective view of a portion of a lateral end of the torque device; and

FIG. 8A-8C are side views of the torque system illustrating various torque device locations.

### DETAILED DESCRIPTION

Referring now to the discussion that follows and also to the drawings, illustrative approaches to the disclosed assemblies are shown in detail. Although the drawings represent some possible approaches, the drawings are not necessarily to scale and certain features may be exaggerated, removed, or partially sectioned to better illustrate and explain the present disclosure. Further, the descriptions set forth herein are not intended to be exhaustive or otherwise limit or restrict the claims to the precise forms and configurations shown in the drawings and disclosed in the following detailed description.

Referring to FIGS. 1 and 2, a torqueing system 100 mounted to an exemplary robotic catheter assembly 102 is illustrated in which an apparatus, a system, and/or method may be implemented according to various exemplary illustrations. Torque system 100 (otherwise referred to as a gripping system) may be configured to aid in the insertion of a medical instrument (e.g., a catheter or guidewire) by actuating two torque devices which may allow continuous and infinite motion for both rotation and insertion of the catheter or related devices. The system 100 may include a first torque device 104 and a second torque device 106 (otherwise referred to as a first gripping device and a second gripping device, respectively). Each of the torque devices 104, 106 may be configured to selectively grip an elongate member such as a medical instrument 114, for example a catheter, microcatheter, guidewire, balloon catheter, sheath, stent, or any other elongate member, merely as examples. The system 100 may permit torqueing or rotation of the instrument 114 by manipulation of the torque devices 104, 106. As described in more detail below, the torque devices 104, 106 may be configured to rotate the instrument 114 and move the instrument 114 axially simultaneously.

System 100 may include a robotic catheter assembly 102 (e.g., a drive mechanism) which may have a first or outer steerable component (e.g., a sheath instrument), hereinafter referred to as a first driver 108, and/or a second or inner steerable component (e.g., a catheter or guide instrument), hereinafter referred to as a second driver 110. First and second driver 108, 110 may be mounted to mounting plates 154, 156, as shown in FIG. 1. First and second driver 108, 110 may be configured to receive first and second torque devices 104, 106, respectively. The various components of the robotic catheter assembly 102 may be controllable by an operator (e.g., physician, surgeon, or practitioner) via an operator workstation (not shown). A surgeon, for example, may sit at the operator workstation and monitor the surgical procedure, patient vitals, and control one or more device. Catheter assembly 102 may be coupled to the workstation via a plurality of cables or other suitable connectors (not shown) to provide for data communication, or one or more components may be equipped with wireless communication components. Communication between components may also be implemented over a network or over the internet.

System 100 may include a driver track 112 on the top portion of the catheter assembly 102 such that first and second driver 108 and 110 may be aligned adjacent to one another within the driver track 112. The first and second driver 108, 110, via the mounting devices 154, 156, may be held to the driver track 112 by a series of articulation mechanisms (not shown), such as shaft pins and alignment pins. The pins may lock shafts that extend from the mounting plates 154, 156 and/or the first and second driver 108, 110 into the driver track 112. During use, drivers 108, 110 may transition along driver track's longitudinal axis, and movement of each driver 108, 110 may be controlled and manipulated independently or in relation to one another. For this purpose, motors, gears, pulleys, and belts within the catheter assembly 102 may be controlled such that carriages coupled to the mounting plates 154, 156 are driven forwards and backwards on bearings, for example.

The drivers 108, 110 may have a predefined or set range of motion on the longitudinal axis while in use. In other words, each driver 108, 110 may be configured to move within a maximum axial stroke length along the driver track 112. For example, first driver 108 may transition along a first portion of the driver track 112, while the second driver 110 may transition along a second portion of the driver track 112 (e.g., the first driver 108 may transition along a first half of the driver track 112 in close proximity to the patient while the second driver 110 may transition along a second half of the driver track 112 farther away from the patient). Although manipulated independently, the drivers 108, 110 may transition along their respective portions of the driver track 112 concurrently or in alternating patterns. That is, while the first driver 108 moves forward, the second driver 110 may remain stationary and while the first driver 108 remains stationary, the second driver 110 may move forward. The converse may also be true when one or both drivers 108, 110 are moving backwards. Further, both drivers 108, 110 may move concurrently forward. Additional motors within the catheter assembly 102 may be activated to control the rotation of the first and second drivers 108, 110 to impart rotational motion to the respective drivers. As a result, an instrument 114 coupled to the drivers 108, 110, via first and second torque device 104, 106, may be controllably manipulated while inserted into the patient. For example, the torque system 100 may be configured such that one torque device grips the instrument 114 and rotates while the other torque device is relaxed (e.g., not gripping the instrument 114 and/or rotating). The gripping torque device may be moved forward via its associated driver while the other torque device may transition backwards or remain stationary via its associated driver. The torque devices may alternate between gripping and rotating such that the torque device gripping and rotating is always moving forward towards the patient. Therefore, the torque devices may continuously insert the instrument into the patient in a “hand-to-hand” arrangement.

With reference to FIGS. 2 and 3, system 100 may include a first torque device 104 and a second torque device 106 positioned over their respective drivers 108, 110. Additionally or alternatively, the system 100 may provide for the first and second torque device 104, 106 to be incorporated into the first and second driver 108, 110, respectively. In general, the torque devices 104, 106 may be configured to removably connect to the respective drivers 108, 110 and be configured to receive an instrument 114. Each torque device may include a housing 120, 140 having an outer periphery 164, 166. The housing 120, 140 may be foamed as a unitary body configured in a cylindrical or barrel shape. The housing 120, 140 may be constructed, for example, by injection molding of polycarbonate, polyethylene terephthalate, high-density polyethylene, low-density polyethylene, polyvinyl chloride, polyetheretherketone, polypropylene, or similar material such that the housings may be produced in high volumes. Alternatively, there may be different molding sizes to fit different lines of torque devices. The housings 120, 140 may be manufactured in a manner such that the outer periphery 164, 166 contains uniform dimensions (e.g., all the housings have a standard size), thus allowing the housings to be interchangeable amongst various catheter assemblies 102. The outer periphery 164, 166 may be ribbed to provide an ergonomic grasping surface or may be smooth to provide an aerodynamic or streamlined surface. Additionally, the material used to manufacture the housings 120, 140 may allow the devices 104, 106 to be disposed of after use. Additionally or alternatively, the devices 104, 106 may be sterilized and reused. The housing 120, 140 may contain within it a radio-frequency identification (RFID) chip which may help the operator workstation determine what device is connected to the robotic catheter assembly 102.

Each housing 120, 140 may include a lumen 130, 132 (e.g., a first lumen 130 and a second lumen 132) configured to receive the instrument 114. The lumen 130, 132 may be at least partially defined by a lumen wall 160, 162 that may constitute a linear tube extending the length of the respective torque devices 104, 106. The lumen wall 160, 162 may extend substantially parallel to the driver track 112. Alternatively, the lumen wall 160, 162 may define a lumen 130, 132 having a frustro-conical or tear-drop shape to facilitate insertion of the instrument 114. The lumen wall 160, 162 may have a flexible inwardly facing diameter or surface configured to compress or expand to facilitate in gripping the instrument 114 (e.g., forming a compressible tube). The flexible material may include any plastic or rubber that is pliable and/or durable and facilitates in clutching or grasping the instrument 114, including, but not limited to, silicone, polyurethane, pebax, etc. In particular, and as will be illustrated below, the flexible inwardly facing surface of the lumen wall 160, 162 may grip the instrument 114 in response to a grip signal and release the instrument 114 in response to a release signal (e.g., selectively grip the instrument 114). For example, as each lumen wall 160, 162 is compressed, the lumen wall 106, 162 may bulge or swell causing the lumen 130, 132 to constrict around the instrument 114. By gripping the instrument 114, the lumen 130, 132, via the lumen wall 160, 162 (and consequently the torque device 104, 106), may become fixedly secured to the instrument 114 in an engaged state. Conversely, by releasing the instrument 114, the lumen wall 160, 162 relaxes to allow each torque device 104, 106 to freely move about the instrument 114 in a released state. The lumen wall 160, 162 may, for example, include a textured surface, e.g., a knurled or diamond-shape texture, for additional friction while gripping the instrument 114.

The housing 120, 140 may include an actuator 116, 138 configured to receive the grip signal to prompt the lumen 130, 132, via the lumen wall 160, 162, to selectively grip the instrument 114. The actuator 116, 138 may be any device configured to trigger the lumen wall 160, 162 to compress or release the instrument 114. For example, a doctor may press or squeeze a button that compresses the lumen wall 160, 162 to grip the instrument 114. The actuator 116, 138 may surround each lumen 130, 132 as a unitary body (e.g., in the form of a washer), or may be on a side of each lumen 130, 132 (e.g., in the form of a lever). The actuator 116, 138 may be press-to-grip, in which the actuator 116, 138 engages the lumen walls 160, 162 to compress when a force acts on the actuator, depressing it, or may be press-to-release in which the actuator engages the lumen walls 160, 162 to compress when the actuator is released. The actuator 116, 138 may be configured to lock in place when pressed (which may be axially or radially), or may require a constant force to remain actuated. The actuator 116, 138 may be located on the end or side of the housing, as shown in FIG. 2. Alternatively, each actuator 116, 138 may be configured on the outer periphery 164, 166 of the respective housings 120, 140.

A gear 118, 142, as shown in FIGS. 3, 4, and 7, may be built into each housing 120, 140 of the torque device 104, 106 so that each torque device 104, 106 may be rotated independently. The gears 118, 142 may be configured to rotate the respective torque device 104, 106 upon receiving an activation signal. The activation signal may be any external source exerting a rotational force on the gear. The gears 118, 142 may be any toothed gear configured to mesh with a corresponding driver associated with the drivers 108, 110. For example, the drivers 108, 110 may each have gears 126, 146 configured to interface with the gears 118, 142, respectively. Merely as examples, the gears 118, 142 may include internal and external spur gears, beveled and spiral beveled gears, spiral or helical gears, worm gears, or any other gear driving mechanism that is convenient. The gears 118, 142 may be configured on the side of each housing 104, 106 opposite the actuator 116.

With specific reference to FIG. 2, the torque system 100 may be positioned over and coupled to the robotic catheter assembly 102 via the first and second drivers 108, 110. The first torque device 104 may couple to the first driver 108 via a first interface 150. The second torque device 106 may couple to the second driver 110 via a second interface 152. The drivers 108, 110 and associated interfaces may be configured to continuously rotate and insert an instrument 114 by way of the torque devices 104, 106. In an exemplary illustration, each driver 108, 110 may have a standard interface 150, 152 that matches the dimensions of the outer periphery 164, 166 of each housing such that a series of interchangeable torque devices may be coupled to the catheter assembly 102 regardless of the size or type of instrument 114 used in a medical procedure. Accordingly, each driver interface 150, 152 may include an actuator trigger 124, 144 and a gear driver 126, 146.

The actuator trigger 124, 144 may be configured to engage the actuators 116, 138 of the respective torque devices 104, 106. The actuator trigger 124, 144 may embody any type of device configured to communicate a grip or release signal to the torque device. For example, the actuator trigger 124, 144 may engage the actuator 116, 138 in a press-to-grip manner. While engaging the actuators 116, 138 to trigger the lumen wall 160, 162 to grip the instrument 114, at least one of the torque devices 104, 106 (or both) may be configured to move forward or backward for effecting a linear motion of the instrument 114, e.g., to insert or withdraw the instrument 114 from a patient, respectively. As depicted in FIG. 2, the actuator trigger 124, 144 may exemplify a lever assembled to move laterally or pivot in order to trigger the actuator. For example, the actuator trigger 124, 144 may exert a constant force on the actuator 116, 138 to trigger and maintain gripping of the lumen wall 160, 162 on the instrument 114. Additionally or alternatively, the actuator trigger 124, 144 may exert an initial force on the actuator 116, 138 to lock the actuator 116, 138 in place to maintain gripping of the lumen wall 160, 162 in the engaged state. In the former example, the instrument 114 may be released from the lumen 130, 132 by retracting the actuator trigger 124, 144 to break the force exerted on the actuator 116, 138 thereby allowing the actuator 116, 138 to extend to its original position. In the later example, the actuator trigger 124, 144 may exert a second force on the actuator 116, 138 to release the actuator 116, 138 from the locked position.

The gear drivers 126, 146 may be configured to engage the gears 118, 142 of the respective torque devices 104, 106 to facilitate rotation of the devices 104, 106. As mentioned previously, gear drivers 126, 146 may be configured to mesh with their corresponding gears 118, 142 located on the torque devices 104, 106. Each gear 118, 142 may receive an independent activation signal from the gear driver 126, 146 directing the gear 118, 142 to rotate their respective housings 120, 140. The gear drivers 126, 146 may control the rotational speed of each torque device 104, 106 independently. As a result, one torque device may rotate at a slower or faster rate than the other torque device. Both actuator triggers 124, 144 and gear drivers 116, 138 may be coupled to a motor within their respective drivers 108, 110 to accomplish their particular functions. Additionally or alternatively, each driver 108, 110 may include a support 128, 148 located next to the gear drivers 126, 148 to help position the torque devices 104, 106. The support 128, 148 may serve to secure the torque devices 104, 106 in place as well as serve as a backstop to oppose the actuator trigger 124, 144 while it is exerting force on the torque devices 104, 106.

When an instrument 114 is prepared for use with the torque system 100, the instrument 114 may be threaded through each lumen 130, 132 of the first and second toque device 104, 106. That is, the instrument 114 may first be inserted into the lumen 130 of the first torque device 104 such that a proximal position (in relation to the patient) or first portion of the instrument 114 lies within the first torque device 104. The instrument may be extended through the lumen 130 of the second torque device 106 such that a distal or second portion of the instrument 114 lies within the second torque. The first and second torque device 104, 106 may be configured such that the lumen wall 160, 162 of each is in a relaxed state to allow the instrument 114 to be easily inserted and extended through each lumen 130, 132. Additionally, the first and second torque device 104, 106 may be aligned spatially adjacent to one another along a linear axis. In an exemplary illustration, an anti-buckling device 122 may be arranged between the first torque device 104 and the second torque device 106 to encompass the instrument 114 providing lateral support to the instrument 114. The anti-buckling device 122 may limit the motion of the instrument 114 to one degree of freedom. Additionally or alternatively, the anti-buckling device 122 may connect the first torque device 104 to the second torque device 106. The anti-buckling device 122 may expand or contract as the first and second torque device 104, 106 transitions along the linear axis.

As explained previously, the system 100 may be mounted onto the robotic catheter assembly 102 via the first and second interface 150, 152 of each respective driver 108, 110, as shown in FIGS. 1 and 2. The first and second torque device 104, 106 may be received by their corresponding drivers 108, 110 either before or after each torque device 1104, 106 receives the instrument 114. This configuration may allow for the entire torque system 100 to be top-loaded onto the robotic catheter assembly 102 as a single package (e.g., installing the torque system 100 from overhead). Having a standard driver interface 150, 152 along with identical outer peripheries 164, 166 for all torque devices provides for a secure connection and interchangeability between the torque devices 104, 106 and their associated drivers 108, 110. Additionally, a drape (e.g., a sterile sheet, such as a plastic sheet) may be inserted either between the torque devices 104, 106 and the drivers 108, 110, or between the drivers 108, 110 and the driver track 112 in order to keep the draped components, e.g., of the robotic catheter assembly 102, out of the sterile environment during use. For example, the drape may create a sterile barrier between the first and second gripping devices 104, 106 (or torque devices) and the drive mechanism. A splayer (not shown) may be incorporated into, or placed on top of, the first and second driver 108, 110. The splayer may function to connect and lock the robotic catheter assembly 102 to the instrument 114. Additionally or alternatively, a splayer cover (not shown) may be fixably coupled to the first and second driver locking each torque device 104, 106 into place. For example, a splayer cover may swing or be placed over the torque devices 104, 106 to lock them in place on their respective drivers 108, 110 (e.g., by a magnetic latching assembly). An advantage of a top-loading configuration for the torque devices 104, 106 may be to allow for the operator to quickly and easily interchange a different torque device 104, 106, instrument 114, or entire torque system 100. For example, some medical procedures may require use of multiple instruments 114 (e.g., catheters, guide wires, etc.). Because medical personnel often exchange out instruments 114 that are as long as 300 centimeter (cm), removing and exchanging torque devices 104, 106 may increase the medical procedure's duration. By providing a standard driver interface 150, 152, various torque devices 104, 106 receive the various instruments 114 at once and then may be top-loaded onto the robotic catheter assembly 102 when needed. As a result, workflow (e.g., replacing and exchanging torque devices 104, 106 and instruments 114) may be significantly improved and may decrease surgery duration accordingly.

As, explained, the torque system 100 may be configured to enable one torque device to be in an active state (e.g., performing the gripping and rotating function) while the other torque is in a passive state. That is, the torque devices 104, 106 may function as a hand-to-hand feature to continuously propel the instrument 114. For example, the torque system 100 may provide that at least one torque device 104, 106 is gripping the instrument 114 in an engaged state at all times. The actuator 116 of first torque device 104 may receive a signal from the actuator trigger 124 to compel the first lumen 130, via the lumen wall 160, to grip the instrument 114, thus securing the instrument 114 in an engaged state. Simultaneously, the gear 118 of the first torque device 104 may receive an activation signal from the gear driver 126 to engage the gear 118 and effect rotation of the torque device 104. Initiation of the engaged state may trigger the activation signal, thereby effecting simultaneous gripping of the instrument 114 and rotation of the torque device 104, 106. The first driver 108 may propel the first torque device 104 forward towards the patient, thereby causing the instrument 114 to insert into the patient (along with simultaneous rotation). The second torque device 106, on the other hand, may be in the passive state such that the neither the lumen 132 is in the engaged state (e.g., the lumen wall 162 is in the released state) nor the housing 140 in rotation.

Each torque device 104, 106 may be configured to travel over a maximum axial stroke length. For example, each torque device 104, 106 may have a predefined range of motion along the axis on which it can travel. Once the first torque device 104 approaches the end of its predefined range of motion (e.g., its maximum axial stroke length) along the longitudinal axis, the second torque device 106 may be configured to switch to the active stage, assuming control and insertion of the instrument 114 (e.g., the second lumen 132 of the second housing 140 is in the engaged state and the torque device 106 is rotating while the torque device is moving forward). At the same time, the first torque device 104 may switch to the passive stage and retract to its original position at the beginning of the longitudinal axis. Each torque device 104, 106 may be capable of moving forward and backward along robotic catheter assembly 102 independently regardless of whether it is in the active or passive state. Accordingly, the first and second torque devices 104, 106 may be configured to cooperate to continuously grip the instrument 114 while simultaneously moving the instrument 114 through a first distance axially with respect to the housing. By working in cooperation, the first and second torque device 104, 106 may move the instrument over a distance greater than the maximum axial stroke length of each respective torque device 104, 106. Configuring the torque system 100 to provide for one of the torque device to be active at all times, the torque system 100 may achieve continuous and infinite insertion and rotation by alternating passive and active duties between each torque device and allowing each torque device to reset before assuming the active duty. Continuous rotation may be desirable in order to decrease friction while inserting the instrument into the patient. Additionally or alternatively, the torque system 100 may be configured to provide that both torque devices 104, 106 be in the active state at all times. This may be desired where extra force is necessary for insertion of the instrument 114 into the patient over distances within the range of motion of the drivers 108, 110.

FIGS. 3 and 4 show an exemplary torque device 104, 106. The torque device 104, 106 and associated components (e.g., actuator 116, housing 120, and gear 118) may form a standardized outer canister 158 having a consistent size or interface, despite any difference in the internal components thereof. By forming a canister 158 that complies with existing standards, the torque devices 104, 106 may be easily accepted by the drivers 108, 110 of the catheter assembly 102. Moreover, the torque devices 104, 106 may be easily interchanged and disposed of during or after a medical procedure. Additionally, multiple instruments of varying diameters may also be accepted by the torque devices 104, 106. As shown by way of example in FIG. 3, the lumen 130 may receive a small instrument 114 while FIG. 4 illustrates the lumen 130 receiving a larger instrument 114. Examples of a small instrument 114 may include a microcatheter or guidewire. Examples of a large instrument may include a balloon catheter or stent. Likewise, a standard outer canister 158 may additionally allow for the robotic catheter assembly 102 to include a standard interface 150, 152 (as shown in FIG. 2) common to all torque devices 104, 106 while allowing the lumens 130, 132, via the lumen wall 160, 162, to grip a wide variety of instruments 114 that may be used by the robotic catheter assembly 102. Thus, the system provides for flexibility and interchangeability. As illustrated in FIG. 3, the torque device 104, 106 having a standard outer canister 158 may be adjusted to grip a small instrument. In this example, the lumen wall 160, 162 may initially be in a relaxed or released state in order to allow the lumen 130, 132 to slidably receive the instrument 114. Generally, a grip signal may be received by the actuator 116 to adjust the lumen wall 160, 162 to the size of the instrument 114. Likewise, FIG. 4 illustrates a torque device 104, 106 also configured to receive an instrument 114. In this example, the instrument 114 is larger than that shown in FIG. 3. The outer canister 158, however, has the same dimensions as the outer canister 158 illustrated in FIG. 3 holding the smaller instrument 114. Accordingly, instruments with varying diameters may be received by the torque device 104, 106 while maintaining a standard outer canister 158 configured to be received by the drivers 108, 110.

FIGS. 5A and 5B represent cross-sectional views of the torque device 104, 106 having a released and depressed actuator 116, respectively. Each lumen 130, 132 may be defined by a lumen wall 160, 162 having a flexible diameter or inwardly facing surface defining a passage. In one exemplary configuration, the lumen may form a rubber tube. The lumen wall 160, 162 may be configured to compress and grip the instrument 114 in response to a grip signal and may expand to release the instrument 114 in response to a release signal. For example, the lumen 130, 132, via the lumen walls 160, 162, may be in a normally open state. That is, the lumen walls 160, 162 may initially be relaxed allowing the instrument to easily slide into the torque device 104, 106. Upon a grip signal, the lumen walls 160, 162 may compress onto the instrument 114. Alternatively, the lumen 130, 132, via the lumen walls 160, 162, may be in a naturally closed state. The lumen walls 160, 162 may initially be compressed, requiring an actuation signal to open the lumen 130, 132 for insertion of the instrument 114. For example, an actuation signal may trigger the lumen walls 160, 162 to open for insertion of the instrument 114 (e.g., a practitioner pressing the actuator 116, 138 to open the lumen walls 160, 162), and then the lumen walls 160, 162 may compress automatically upon release of the actuation signal (e.g., the practitioner lets go of the actuator 116, 138).

FIG. 5A shows a section view of the exemplary torque device 104, 106 in a released state. In this state, the lumen wall 160, 162 may be relaxed to allow the instrument 114 to be easily inserted into the lumen 130, 132 of each torque device 104, 106. As such, the actuator 116 is disengaged or is in a non-depressed state such that the flexible diameter of the lumen wall 160, 162 may be fully extended. Once the instrument 114 has been inserted through the lumen 130, 132, the actuator 116, 138 may receive a grip signal from the interface 150, 152 of the first or second driver 108, 110 triggering the actuator to depress and constrict the flexible diameter of the lumen wall 160, 162. Additionally or alternatively, grip signal may be in the form of an operator, e.g., a surgeon, squeezing or pressing the actuator 116, 138 which may compress the flexible diameter of the lumen wall 160, 162. As the actuator 116, 138 is depressed, the lumen wall 160, 162 is forced to engage and constrict upon the instrument 114 increasingly imparting more grip friction and force onto the instrument 114. As shown in FIG. 5B, a bulge is created within the center of the lumen 130, 132 that clamps onto the instrument 114 to create a firm, frictional engagement with the instrument 114. That is, the actuator 116, 138 may press against the lumen wall 160, 162 causing the flexible diameter of the lumen wall 160, 162 to swell and bulge into the open lumen 130, 132 passageway. The lumen wall 160, 162 may bulge on either end of the lumen 130, 132 creating two points of contact with the instrument 114. Alternatively, the bulge may circumscribe the entire circumference of the lumen 130, 132, creating an enlarged contact patch between the lumen wall 160, 162 and the instrument 114. Accordingly, the lumen wall 160, 162 may apply a generally constant and even pressure along the instrument 114 so as to not crush or flatten the instrument 114. The swelling of the lumen wall 160, 162 as it presses onto the instrument 114 from all directions creates a tight and even grip across a relatively large contact patch, which may increase the grip of the torque device's hold on the instrument 114. This engagement is referred to herein as an engaged state. In order to release the instrument 114, the actuator 116, 138 may be disengaged, allowing the lumen 130, 132 to extend and relax in a released state.

Additionally or alternatively, the flexible diameter of the lumen wall 160, 162 may only define a portion of the lumen 130, 132 passage. For example, the flexible diameter of the lumen wall 160, 162 may constitute a donut or washer shaped structure abutting each actuator 116, 138 that may bulge upon depression of the actuator 116, 138. The lumen 130, 132 has been described as having a lumen wall 160, 162 with a flexible diameter (e.g., a rubber tube) that bulges or balloons when compressed by the actuator 116, 138. Additional and alternative examples may be employed consistent with this disclosure.

The force or pressure applied on the instrument 114 by the lumen wall 160, 162 in the engaged state may be predefined based on the tensile and compression strength of the instrument 114 being used. For example, the pressure applied to the actuator 116, 138 forcing the lumen wall 160, 162 to compress and grip the instrument 114 may be less for a catheter as compared to a guidewire. Information relating to the strength of various instruments 114 may be stored within the operator workstation. Depending on the instrument 114 being used, the gripping force of the lumen wall 160, 162 in the engaged state may not exceed the stored (or predefined) threshold for that instrument 114. Accordingly, the balance between grip friction and force exerted on the instrument 114 may be customized to avoid crushing or flattening of the instrument 114.

Referring to FIGS. 6A and 6B, an illustrative torque device 600 may have connected housings 602 and 604. As illustrated, the torque device 600 may include two distinct housings 602, 604 coupled together at a medial connection 610 and adjoined by gears 606, 608 on their respective lateral ends. Additionally or alternatively, the gears 606, 608 may be coupled to the torque device 600 medially, as shown in FIG. 6B. The gears may be rotated in opposing directions in order to tighten and release the lumen's grip of the instrument 114. For example, as one gear 606 and the respective housing 604 is rotated clockwise, the other gear 608 and housing 602 may be rotated counterclockwise in order to tighten the grip on the instrument 114. The lumen 130, 132 may consist of a lumen wall 160, 162 having an axial slit 612 running the width of the lumen wall 160, 162 in order to compensate for the tightening and releasing function of the housing. The slit may expand and contract to accommodate the size of the instrument 114. That is, as the torque device 600 tightens its' grip on the instrument (e.g., by rotating the gears 606, 608 in opposite directions), the two edges bordering the slit may overlap to form a more compact fit on the instrument 114.

FIG. 7 illustrates a perspective view of a portion of a lateral end of the torque device 104, 106. As shown, the gear 118, 142 may connect to the housing 120, 140 of each torque device 104, 106. The gear 118, 142 may be secured to each housing 118, 142 via a fastener 134. For example, protuberances may be molded into the gear and fit into a depression 136 within the housing. Additionally or alternatively, the fastener 134 may be a screw or bolt that is inserted into the depression 136 to connect the gear 118, 142 to the housing 120, 140.

The torque devices 104, 106 may be configured to rotate with respect to the housing 120, 140 over a maximum radial stroke angle. For example, each torque device 104, 106 may have a defined angle on which it may be rotated. The first and second torque device 104, 106 may work together or cooperate in order to alternate rotation of the instrument 114. For example, the responsibility of rotating the instrument 114 may alternate between the first and second torque devices 104, 106. Cooperation between the first and second torque device 104, 106 may allow the instrument 114 to be rotated at an angle greater than the maximum radial stroke angle.

Referring now to FIGS. 8A-8C, the torque system 100 may be configured to allow for infinite and continuous insertion and rotation of the instrument 114 by the cooperation of the torque devices 104, 106, e.g., in an alternating fashion. Each torque device 104, 106 may be capable of moving forward and backward independently (e.g., in a first direction for insertion and second direction in opposition to the first, e.g., for withdrawal) and the torque devices may alternate between an engaged and a disengaged state such that at least one of the devices is in an engaged state at all times. With specific reference to FIG. 8A, the two torque devices 104, 106 positioned close to each other (e.g., a first position). The first torque device 104 may initially receive a grip signal triggering the first lumen wall 160 to grip the instrument 114 in an engaged state. Additionally, the gear 118 may receive an external activation signal (e.g., engagement by the first gear driver 126) that rotates the first torque device 104 and, consequently, the instrument 114 secured within the first lumen 130. The second torque device 106, on the other hand, may be in a released state such that the second lumen wall 162 is released and relaxed from the instrument 114. The torque devices 104, 106 may then move away from one another. The first torque device 104 may transition forward while in the engaged state providing for insertion and rotation of the instrument 114 into the patient. The second torque device 106 may transition backward (e.g., in an opposite direction from the first torque device 104) such that the first and second torque device 104, 106 are moving away from one another along a linear axis.

Referring to FIG. 8B, as each torque device 104, 106 reaches or nears the end of their respective predefined range of motion (e.g., such that first toque device 104 is further spaced from the second torque device 106), the second torque device 106 may engage the instrument 114 in a second position and begin rotating and inserting while the first torque device 104 may release the instrument 114. Additionally or alternatively, the second torque device 106 may begin rotating and inserting prior to engaging the instrument 114 in order to catch-up to the rotational and/or insertion speed of the first torque device 104 to provide for a smooth changeover. Once the rotational and insertion speeds have been matched between the first and second torque device 104, 106, the second torque device 106 may secure the instrument 114 in the engaged state to begin inserting the instrument 114 towards the patient. The changeover may be configured to occur in a seamless fashion, such that there is no break in the insertion or rotation of the instrument 114 into the patient. As the second torque device 106 begins inserting, the first torque device 104 may release the instrument 114 and begin to withdrawal to its original starting position at the beginning of the first torque device's predefined range of motion. Accordingly, the second torque device 106 may be transitioning forward (e.g., inserting) as the first torque device 104 withdrawal's backwards such that the two torque devices 104, 106 move in a direction converging towards one another.

Referring to FIG. 8C, as each torque device 104, 106 transitions to the first position near one another, the first torque device 104 may engage, rotate, and insert the instrument 114 towards the patient while the second torque device 106 releases the instrument 114. As previously mentioned, the first torque device 104 may begin to rotate and insert prior to the changeover. The first and second torque device 104 and 106, therefore, may work in cooperation to continuously grip, rotate, and move the instrument 114 axially with respect to the housing 120, 140.

Thus, torque system 100 may be configured such that as one torque device grips the instrument and begins inserting and rotating towards the patient, the other torque device moves back to its original range of motion on the linear axis in a released state. As the engaged torque device nears the end of its range of motion, the released torque device may begin to match to rotational and insertion speed of the engaged torque device, and then engage the instrument itself while the other torque releases the instrument and resets to its original starting position on the linear axis. In other words, the process may essentially be described as a simple hand to hand pulling motion to continuously insert the instrument into the patient.

The exemplary illustrations are not limited to the previously described examples. Rather, a plurality of variants and modifications are possible, which also make use of the ideas of the exemplary illustrations and therefore fall within the protective scope. Accordingly, it is to be understood that the above description is intended to be illustrative and not restrictive.

In general, computing systems and/or devices such as such as the controllers, biometric devices, displays telematics functions, etc., may employ any of a number of computer operating systems, including, but by no means limited to, versions and/or varieties of the Microsoft Windows® operating system, the Unix operating system (e.g., the Solaris® operating system distributed by Oracle Corporation of Redwood Shores, Calif.), the AIX UNIX operating system distributed by International Business Machines of Armonk, N.Y., the Linux operating system, the Mac OS X and iOS operating systems distributed by Apple Inc. of Cupertino, Calif., the BlackBerry OS distributed by Research In Motion of Waterloo, Canada, and the Android operating system developed by the Open Handset Alliance.

Computing devices, such as the controllers, biometric devices, displays telematics functions, etc., may generally include computer-executable instructions that may be executable by one or more processors. Computer-executable instructions may be compiled or interpreted from computer programs created using a variety of programming languages and/or technologies, including, without limitation, and either alone or in combination, Java™, C, C++, Visual Basic, Java Script, Perl, etc. In general, a processor or microprocessor receives instructions, e.g., from a memory or a computer-readable medium, etc., and executes these instructions, thereby performing one or more processes, including one or more of the processes described herein. Such instructions and other data may be stored and transmitted using a variety of computer-readable media.

A computer-readable medium (also referred to as a processor-readable medium) includes any non-transitory (e.g., tangible) medium that participates in providing data (e.g., instructions) that may be read by a computer (e.g., by a processor of a computing device). Such a medium may take many forms, including, but not limited to, non-volatile media and volatile media. Non-volatile media may include, for example, optical or magnetic disks and other persistent memory. Volatile media may include, for example, dynamic random access memory (DRAM), which typically constitutes a main memory. Such instructions may be transmitted by one or more transmission media, including coaxial cables, copper wire and fiber optics, including the wires that comprise a system bus coupled to a processor of a computer. Common forms of computer-readable media include, for example, a floppy disk, a flexible disk, hard disk, magnetic tape, any other magnetic medium, a CD-ROM, DVD, any other optical medium, punch cards, paper tape, any other physical medium with patterns of holes, a RAM, a PROM, an EPROM, a FLASH-EEPROM, any other memory chip or cartridge, or any other medium from which a computer can read.

Databases, data repositories or other data stores described herein may include various kinds of mechanisms for storing, accessing, and retrieving various kinds of data, including a hierarchical database, a set of files in a file system, an application database in a proprietary format, a relational database management system (RDBMS), etc. Each such data store is generally included within a computing device employing a computer operating system such as one of those mentioned above, and are accessed via a network in any one or more of a variety of manners. A file system may be accessible from a computer operating system, and may include files stored in various formats. An RDBMS generally employs the Structured Query Language (SQL) in addition to a language for creating, storing, editing, and executing stored procedures, such as the PL/SQL language mentioned above.

With regard to the processes, systems, methods, heuristics, etc. described herein, it should be understood that, although the steps of such processes, etc. have been described as occurring according to a certain ordered sequence, such processes could be practiced with the described steps performed in an order other than the order described herein. It further should be understood that certain steps could be performed simultaneously, that other steps could be added, or that certain steps described herein could be omitted. In other words, the descriptions of processes herein are provided for the purpose of illustrating certain embodiments, and should in no way be construed so as to limit the claims.

Accordingly, it is to be understood that the above description is intended to be illustrative and not restrictive. Many embodiments and applications other than the examples provided would be apparent upon reading the above description. The scope should be determined, not with reference to the above description, but should instead be determined with reference to the appended claims, along with the full scope of equivalents to which such claims are entitled. It is anticipated and intended that future developments will occur in the technologies discussed herein, and that the disclosed systems and methods will be incorporated into such future embodiments. In sum, it should be understood that the application is capable of modification and variation.

All terms used in the claims are intended to be given their broadest reasonable constructions and their ordinary meanings as understood by those knowledgeable in the technologies described herein unless an explicit indication to the contrary in made herein. In particular, use of the singular articles such as “a,” “the,” “said,” etc. should be read to recite one or more of the indicated elements unless a claim recites an explicit limitation to the contrary. Additionally, use of adjectives such as first, second, etc. should be read to be interchangeable unless a claim recites an explicit limitation to the contrary.

1. A gripping device comprising:
a housing having an outer periphery; and
a lumen wall arranged within the housing, the lumen wall defining a passage, the passage configured to receive an instrument, wherein the lumen wall has a flexible inwardly facing surface configured to grip the instrument in response to receiving a grip signal and release the instrument in response to receiving a release signal.

2. The gripping device of claim 1, wherein the gripping of the instrument secures the instrument within the lumen in an engaged state and wherein the releasing of the instrument releases the instrument within the lumen in a released state.

3. The gripping device of claim 2, wherein the lumen wall applies a pressure to frictionally engage the instrument in the engaged state, wherein the pressure does not exceed a predefined threshold.

4. The gripping device of claim 3, wherein the predefined threshold is defined by the strength of the instrument.

5. The gripping device of claim 1, wherein the lumen wall includes a compressible tube configured to engage the instrument.

6. The gripping device of claim 5, wherein at least one of the grip signal and the release signal is received from an interface in communication with the housing.

7. The gripping device of claim 6, further comprising an actuator configured to receive the at least one of the grip signal and the release signal, wherein the actuator is configured to compress the tube in response to the grip signal.

8. The gripping device of claim 1, further comprising a gear configured to rotate a torque device in response to an activation signal.

9. An elongate device drive mechanism comprising:
a first gripping device having a first housing and a first lumen arranged within the first housing, the first lumen configured to receive a first portion of an instrument;
a second gripping device having a second housing and a second lumen arranged within the second housing, the second lumen configured to receive a second portion of the instrument, wherein the second gripping device is spaced and moveable along an axis with respect to the first gripping device; and
wherein each of the first lumen and the second lumen include a lumen wall with a flexible inwardly facing surface configured to selectively grip the respective portion of the instrument.

10. The drive mechanism of claim 9, wherein each lumen wall of the gripping devices is configured to grip the instrument in response to receiving a grip signal and release the instrument in response to receiving a release signal.

11. The drive mechanism of claim 10, wherein the gripping of the instrument secures the instrument within the respective lumen wall in an engaged state and wherein the releasing of the instrument releases the instrument in a released state.

12. The drive mechanism of claim 9, wherein the first and second gripping devices are configured to travel axially with respect to the first and second housings, respectively, over a maximum axial stroke length; and wherein the first and second gripping devices are configured to cooperate to continuously grip the instrument while simultaneously moving the instrument through a first distance axially with respect to the housing, the first distance greater than the maximum axial stroke length.

13. The drive mechanism of claim 12, wherein the gripping devices are configured to alternate between a near and far position with respect to each other, and in each of the positions, one of the lumen walls of a respective gripping device is in an engaged stated and the other lumen wall of the other gripping device is in a released state.

14. The drive mechanism of claim 9, wherein the first and second gripping devices are configured to cooperate to continuously grip the instrument while simultaneously rotating the instrument with respect to the first and second housings, respectively.

15. The drive mechanism of claim 14, wherein the first and second gripping devices are configured to rotate with respect to the first and second housings, respectively, over a maximum radial stroke angle; and wherein the first and second gripping devices are configured to cooperate to continuously grip the instrument while simultaneously rotating the instrument with respect to the first and second housings, respectively, through a first angle, the first angle greater than the maximum radial stroke angle.

16. The drive mechanism of claim 9, wherein the first and second gripping devices are configured to rotate the instrument with respect to the first and second housings, respectively, and simultaneously move the instrument axially with respect to the first and second housings, respectively.

17. The drive mechanism of claim 9, further comprising a disposable portion defining a sterile barrier between the first and second gripping devices and the drive mechanism.

18. The drive mechanism of claim 9, wherein the first housing includes a first gear and the second housing includes a second gear, wherein at least one of the first and second gears is configured to rotate at least one of the respective housing in response to an activation signal.

19. The drive mechanism of claim 18, wherein the activation signal is triggered by a grip signal.

20. The drive mechanism of claim 10, wherein at least one of the grip signal and the release signal is received from an interface in communication with the housing.
